(19) United States
(12) Patent Application Publication
(10) Pub. No.: US 2016/0106400 A1
(43) Pub. Date: Apr. 21, 2016

(54) ENDOSCOPIC SIDE RELEASE BIOPSY VALVE

(71) Applicant: NEW YORK UNIVERSITY, New York, NY (US)

(72) Inventor: Ilseung Cho, New York, NY (US)

(73) Assignee: NEW YORK UNIVERSITY, New York, NY (US)

(21) Appl. No.: 14/883,423

(22) Filed: Oct. 14, 2015

Related U.S. Application Data
(60) Provisional application No. 62/064,160, filed on Oct. 15, 2014.

(51) Int. Cl. A61B 10/04; A61B 1/015

(52) U.S. Cl. CPC A61B 10/04; A61B 1/015

(57) ABSTRACT

A biopsy valve for an endoscope includes a valve body that is laterally divided into an upper portion having a first height and a lower portion having a second height, a through hole provided along a central, longitudinal axis of the valve body, and a tab disposed between the upper portion of the valve body and the lower portion of the valve body. The upper portion and the lower portion are hingedly connected such that the upper portion is configured to rotate about a hinge and move relative to the lower portion, which is generally stationary. The upper portion is biased in a closed position in which suction is maintained in the endoscope. Exerting an upward or a downward force on the tab rotates the upper portion about the hinge into an open position in which suction is released in the endoscope.

### CROSS-REFERENCE TO RELATED APPLICATIONS

This application claims the benefit of U.S. Provisional Application No. 62/064,160 filed on Oct. 15, 2014, which is hereby incorporated by reference in its entirety.

### TECHNICAL FIELD

The present disclosure relates generally to an endoscopic biopsy valve. More specifically, the present disclosure relates to an endoscopic biopsy valve having a side release configured to allow an endoscopist to release the suction without having to remove the biopsy valve or open a top of the biopsy valve.

### BACKGROUND

Endoscopes and biopsy valves are used in every endoscopic procedure, including, but not limited to gastrointestinal endoscopic procedures, bronchoscopic procedures and laparoscopic procedures. The biopsy valve is typically a disposable product that is replaced for every endoscopic procedure. Endoscopic procedures are common procedures. For example, there are over 14 million colonoscopies performed annually in the United States alone.

Referring to FIG. 1, an endoscope 100 generally includes a supply plug 10, an umbilical cord 20, a control head 30, an insertion tube (shaft) 40, and a bending section 50. The supply plug 10 is located at one end of the umbilical cord 20 and includes a plurality of connectors 11. The connectors 11 are configured to connect the endoscope 100 to a plurality of external elements, including, but not limited to a light guide, an air supply, a water bottle and suction, and an air vent. The umbilical cord 20 connects the supply plug 10 to the control head 30, which is attached to the other end of the umbilical cord 20. The control head 30 is provided between the umbilical cord 20 and the insertion tube 40. The control head 30 includes a plurality of controls for air insufflation, irrigation, suction. The control head 30 may also include mechanisms for steering the bending section 50 located at a distal end of the endoscope 100. The insertion tube 40 has a distal bending section 50 whose tip houses terminal ends of an illuminating end of a light guide, air and water jets, and a common port (i.e., a combined suction and biopsy port), and a distal lens or video chip. As used herein, the terms “port” and “channel” are synonymous.

A fiberoptic endoscope has an eyepiece, while a video endoscope has remote control buttons for a video control unit. In a fiberoptic endoscope, light is conducted from the distal lens in the bending section 50 to the eyepiece by bundles of optical glass fibers. In a video endoscope, the image is captured with the video chip in the bending section 50, transmitted electronically, and displayed on a monitor.

The biopsy port 60 is located at an intersection of the insertion tube 40 and the control head 30. The biopsy port 60 is configured for passing sampling and/or operative instruments down the insertion tube 40 and the bending section 50 to an internal site to be examined or surgically manipulated. Referring now to FIGS. 2 and 3, the biopsy port 60 is covered by a biopsy valve 61 that includes a body 62 and a lid 63 tethered to the body 62. The body 62 has a through hole 64 configured to receive a flange 65 of the lid 63, thereby forming a seal. The lid 63 is configured to be reversibly closed (i.e., a position in which the flange 65 is inserted into the through hole 64) (see FIG. 2) and opened (i.e., a position in which the flange 65 is removed from the through hole 64) (see FIG. 3). The biopsy valve 61 is further configured to close off a proximal opening of the biopsy port 60 and prevent room air from being drawn into a suction port 70 located at a distal end of the control head 30. Opening the lid 63 or completely removing the biopsy valve 61 from the biopsy port opening releases the suction in the biopsy port 60. The lid 63 may be difficult to open during an endoscopic procedure, which may render the suction release technique of removing the lid 63 rarely utilized.

Referring now to FIG. 1, the biopsy port 60 converges with the suction port 70 to form a common port 80 that passes down the insertion tube 40 and terminates at a distal end of the bending section 50. Suction is controlled by a suction valve 31 on the control head 30. The suction valve 31 has two positions: a neutral position and a suction position. When the suction valve 31 is not activated, it automatically assumes a neutral position and blocks suction within the suction port 70 and common port 80. When the endoscopist depresses the suction valve 31, the suction port 70 is opened, creating a negative pressure that draws air or fluid into the terminal end of the common port 80 (located in the bending section 50).

Referring now to FIGS. 4-6, during the course of an endoscopy, mucosa is often suctioned into the suction port 70 and the common port 80. In order to release the mucosa, the biopsy valve 61 has to be removed from the endoscope 100 (FIG. 6) or the lid 63 of the biopsy valve 61 has to be opened (FIG. 5). Removal of the biopsy valve 61 or opening of the lid 63 of the biopsy valve 61 requires the endoscopist to use two hands—one hand to hold the endoscope 100 and the other hand to remove or open the biopsy valve 61. The release of the mucosa is often performed multiple times during a single endoscopy, which decreases the endoscopist's efficiency due to the time spent removing and replacing the biopsy valve 61 or opening and closing the lid 63 of the biopsy valve 61. In addition, when the biopsy valve 61 is removed from the endoscope, fecal or other bodily material is ejected from the uncovered biopsy port 60. This increases the risk of contamination and may pose a risk to the endoscopist and other individuals assisting in the endoscopic procedure.

A need exists for improved technology, including technology that may address the above problems, namely by providing an endoscopic biopsy valve and method of operating an endoscopic biopsy valve that allows an endoscopist to release the suction without having to remove the biopsy valve or open a top of the biopsy valve.

### SUMMARY

One embodiment relates to a biopsy valve for an endoscope that includes a valve body that is laterally divided into an upper portion having a first height and a lower portion having a second height, a through hole provided along a central, longitudinal axis of the valve body, and a tab disposed between the upper portion of the valve body and the lower portion of the valve body. The upper portion and the lower portion are hingedly connected such that the upper portion is configured to rotate about a hinge and move relative to the lower portion, which is generally stationary. The upper portion is biased in a closed position in which suction is maintained in the endoscope. Exerting an upward or a downward force on the tab rotates the upper portion about the hinge into an open position in which suction is released in the endoscope.

Another embodiment relates to an endoscope configured for use in an endoscopic procedure. The endoscope includes a biopsy port, a suction port configured to converge with the biopsy port and maintain suction in the biopsy port, and a biopsy valve configured to close off a proximal opening of the biopsy port and prevent room air from being drawn into the suction port. The biopsy valve includes a valve body that is laterally divided into an upper portion having a first height and a lower portion having a second height, a through hole provided along a central, longitudinal axis of the valve body, and a tab disposed between the upper portion of the valve body and the lower portion of the valve body. The upper portion and the lower portion are hingedly connected such that the upper portion is configured to rotate about a hinge and move relative to the lower portion, which is generally stationary. The upper portion is biased in a closed position in which suction is maintained in the endoscope. Exerting an upward or a downward force on the tab rotates the upper portion about the hinge into an open position in which suction is released in the endoscope.

Yet another embodiment relates to a method of operating a biopsy valve of an endoscope, where the biopsy valve includes a valve body that is laterally divided into an upper portion having a first height and a lower portion having a second height, the upper portion and the lower portion being hingedly connected such that the upper portion is configured to rotate about a hinge and move relative to the lower portion, which is generally stationary, a through hole provided along a central, longitudinal axis of the valve body, and a tab disposed between the upper portion of the valve body and the lower portion of the valve body. The method includes exerting an upward or a downward force on the tab to rotate the upper portion about the hinge from a closed position in which suction is maintained in the endoscope to an open position in which suction is released in the endoscope, and removing the upward or the downward force on the tab to reversibly rotate the upper portion about the hinge to the closed position.

### BRIEF DESCRIPTION OF THE DRAWINGS

The details of one or more implementations of the subject matter described in this specification are set forth in the accompanying drawings and the description below. Other features and aspects of the subject matter will become apparent from the description, the drawings, and the claims presented herein.

FIG. 1 illustrates the parts of an endoscope.

FIG. 2 is a perspective view of a conventional biopsy valve for use in conjunction with the endoscope of FIG. 1, where a lid of the biopsy valve is in a closed position.

FIG. 3 is a perspective view of the conventional biopsy valve of FIG. 2, where the lid of the biopsy valve is in an open position.

FIG. 4 illustrates the endoscope of FIG. 1 in use, where a biopsy port of the endoscope is sealed by the biopsy valve of FIG. 2.

FIG. 5 illustrates the endoscope of FIG. 1 in use, where the lid of the biopsy valve of FIG. 2 is opened to release suction in the biopsy port of the endoscope.

FIG. 6 illustrates the endoscope of FIG. 1, where the biopsy valve of FIG. 2 is detached from the endoscope to release suction in the biopsy port of the endoscope.

FIG. 7 illustrates a first embodiment of a biopsy valve having a valve body divided into an upper portion and a lower portion, where the upper portion is in a closed position to maintain suction in a biopsy port.

FIG. 8 illustrates the biopsy valve of FIG. 7, where a downward force is exerted on a tab to rotate the upper portion into an open position to release suction in the biopsy port.

FIG. 9 illustrates a second embodiment of a biopsy valve having a valve body divided into an upper portion and a lower portion, where the upper portion is in a closed position to maintain suction in a biopsy port.

FIG. 10 illustrates the biopsy valve of FIG. 9, where an upward force is exerted on a tab to rotate the upper portion into the open position to release suction in the biopsy port.

FIG. 11 illustrates the biopsy valve of FIG. 7 from a top view of the endoscope, where a downward force is exerted on the tab to rotate the upper portion into the open position to release suction in the biopsy port.

FIG. 12 illustrates the biopsy valve of FIG. 7 from a side view of the endoscope, where the upper portion is biased in the closed position to maintain suction in the biopsy port.

FIG. 13 illustrates the biopsy valve of FIG. 7 from a top view of the endoscope, where the upper portion is biased in the closed position to maintain suction in the biopsy port.

### DETAILED DESCRIPTION OF VARIOUS EMBODIMENTS

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

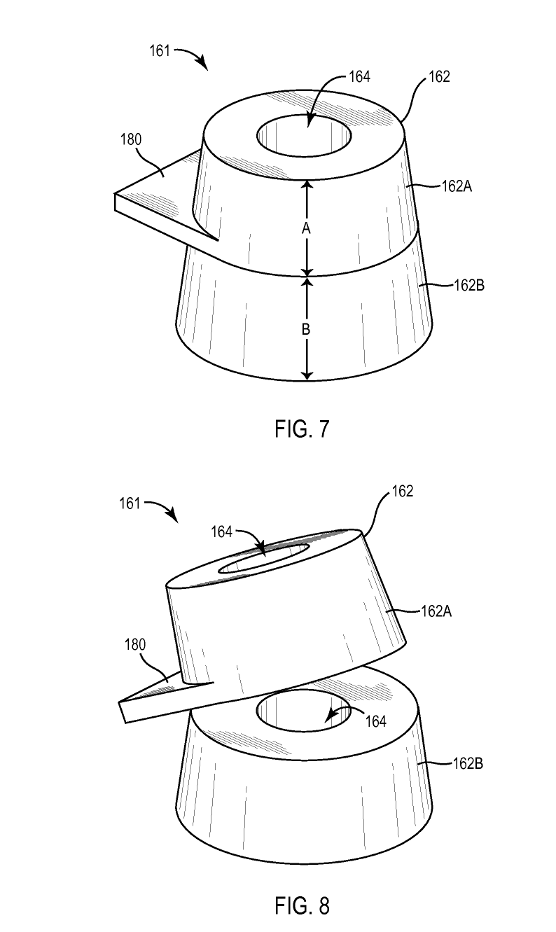

Referring now to FIGS. 7-13, a biopsy valve is configured to provide access for sampling and/or operative instruments to be used in an endoscopic procedure, help minimize leakage of biomaterial from the biopsy port throughout the endoscopic procedure, maintain insufflation and provide access for irrigation. The biopsy valve is configured to removably and reversibly mount on an entrance of the biopsy port of a standard endoscope to provide a seal. The biopsy valve may be reusable or disposable.

### First Embodiment

### Tab Pushed Down

Referring now to FIGS. 7 and 8, a biopsy valve 161 includes a body 162 and a planar tab 180. The biopsy valve 161 may comprise one or more materials that are thermoset or thermoplastic and/or which are elastomeric. In one embodiment, the biopsy valve 161 is made of silicone (or compositions thereof) or thermoplastic elastomer, which exhibits similar elastic properties to silicone. In one embodiment, the body 162 may have a frustoconical shape. In other embodiments, not illustrated, the body 62 may have another shape, for example, a cylindrical shape.

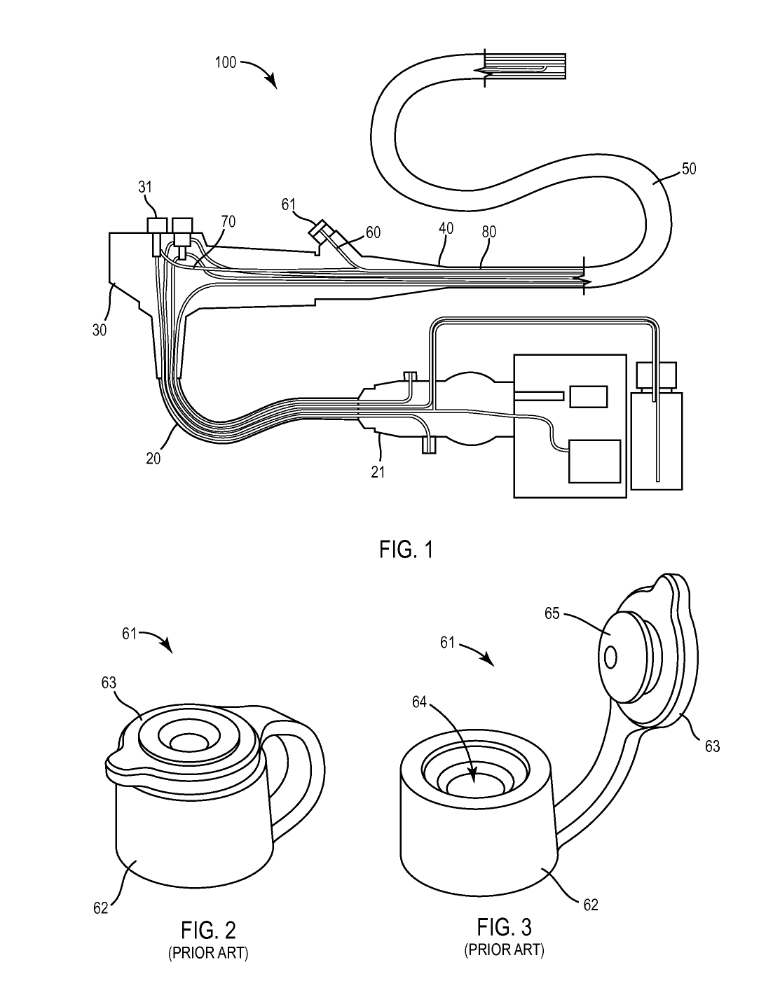

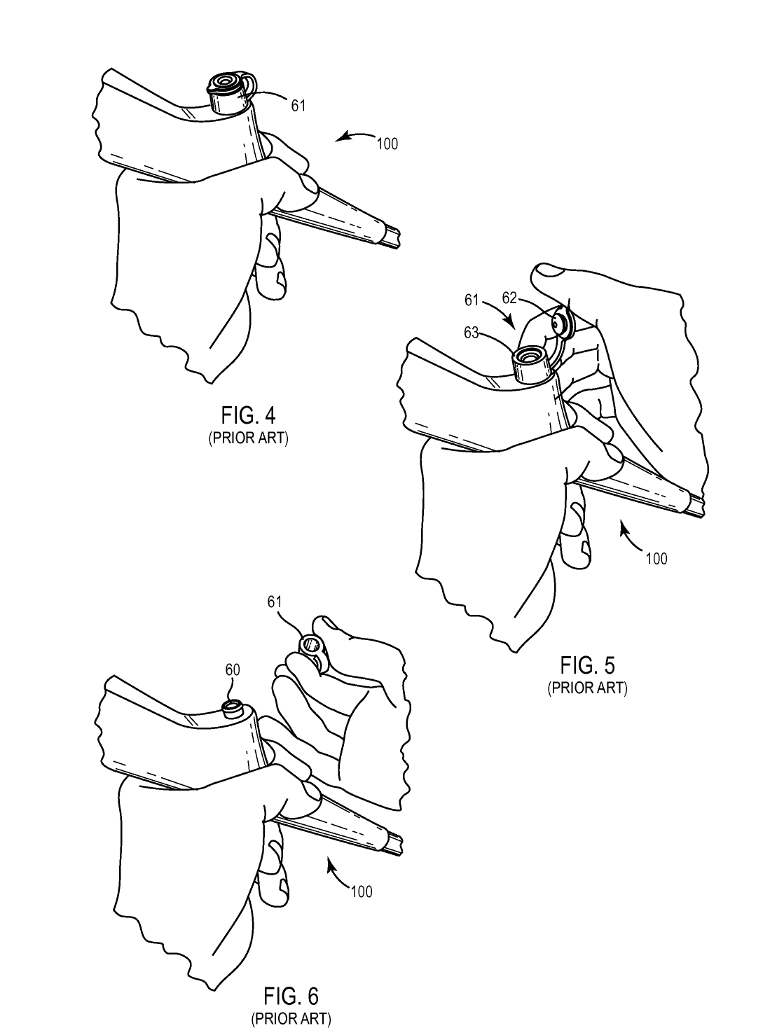

The body 162 is laterally divided into two portions: an upper portion 162A having a height A and a lower portion 162B having a height B. At the location of the perforation that forms the lateral division, a bottom face of the upper portion 162A faces a top face of the lower portion 162B. The upper portion 162A is biased in a closed position (See FIG. 7) by a suction in the biopsy port 60 (see FIG. 1). In some embodiments, the height A and the height B are equal. In other embodiments, the height A is greater than the height B. In additional embodiments, the height A is less than the height B. In other embodiments, the perforation that forms the lateral division may be provided at a predetermined location such that a ratio of the height A to the height B is within a predetermined range. The body 162 further includes a through hole 164 extending along a central, longitudinal axis of the body 162.

The upper portion 162A and the lower portion 162B are hingedly connected, with the upper portion 162A being able to rotate about the hinge and move relative to the lower portion 162B, which is generally stationary. In some embodiments, the biopsy valve 161 includes a tab 180 configured to assist the endoscopist in moving the upper portion 162A from the closed position to an open position (see FIG. 8). When the upper portion 162A is in the open position, the endoscopist can release suction in the biopsy port 60 through a side-vent without removing the entire biopsy valve 161 (see FIG. 6) or opening the lid of the biopsy valve 161 (see FIG. 5).

In order to allow the upper portion 162A to reversibly move between the closed position and the open position, the upper portion 162A and the lower portion 162B are hingedly connected. In one example, the upper portion 162A and the lower portion 162B are hingedly connected by a living hinge provided on a portion of an outer circumference of the upper portion 162A and the lower portion 162B. The tab 180 may protrude through the living hinge such that a first portion of the tab 180 is located at an exterior of the body 162 and a second portion of the tab 180 is located at an interior of the body 162.

In some embodiments, the biopsy valve 161 may include a bi-stable hinge having a thin hinge web having two, spaced apart pivot axes or hinges, one hinge connecting the upper portion 162A to the hinge web and the other hinge connecting the lower portion 162B to the hinge web. The hinge web may be a living hinge. The bi-stable hinge is biased closed and only a small amount of force is needed to shift from a closed position to an open position. The tab 180 may protrude through the bi-stable hinge such that a first portion of the tab 180 is located at an exterior of the body 162 and a second portion of the tab 180 is located at an interior of the body 162.

The tab 180 is configured such that pushing down on the first portion of the tab 180 causes the second portion of the tab 180 to contact and raise the upper portion 162A to an open position. In other words, the tab 180 functions as a lever in that the tab 180 is rigid and pivots at a fixed end or fulcrum (i.e. the hinge connecting the upper portion 162A and the lower portion 162B). Because the upper portion 162A is normally biased in the closed position, once the downward force exerted on the first portion of the tab 180 is removed (i.e., once the endoscopist is no longer pushing down on the first portion of the tab 180), the upper portion 162A returns to the closed position. The upper portion 162A can be reversibly moved from the closed position to the open position, for example, by the endoscopist's thumb such that the endoscopist can use the same hand (i.e., one hand) to operate the endoscope 100 and move the biopsy valve 161 from the closed position to the open position to release suction through the side-vent. This cannot be done for a conventional biopsy valve 61 for which the suction release techniques (illustrated in FIGS. 5 and 6) require two hands to perform.

In one embodiment, the second portion of the tab 180 is only provided in a region proximate to the hinge. For example, the second portion of the tab 180 may extend from the hinge to the through hole 164 extending along the central, longitudinal axis of the body 162. In the case where the second portion of the tab 180 is only provided in the region proximate to the hinge, a slight gap may be formed between the upper portion 162A and the lower portion 162B. In some embodiments, this gap may be eliminated by disposing the second portion of the tab 180 in a recess provided in the bottom face of the upper portion 162A (or, alternatively, in the top face of the bottom portion 162B) such that a bottom face of the second portion of the tab 180 is planar with the bottom face of the upper portion 162A (or, alternatively, such that a top face of the second portion of the tab 180 is planar with the upper face of the lower portion 162B). In other embodiments, the gap may be sealed by a flange protruding from the upper portion 162A or the lower portion 162B.

In another embodiment, the second portion of the tab 180 may have a same surface area as a surface area of the bottom face of the upper portion 162A. In this embodiment, a center of the second portion of the tab 180 includes an aperture having the same dimensions as the through hole 164, such that biopsy forceps or other tools may still be passed through the biopsy port 60.

### Second Embodiment

### Tab Pushed Up

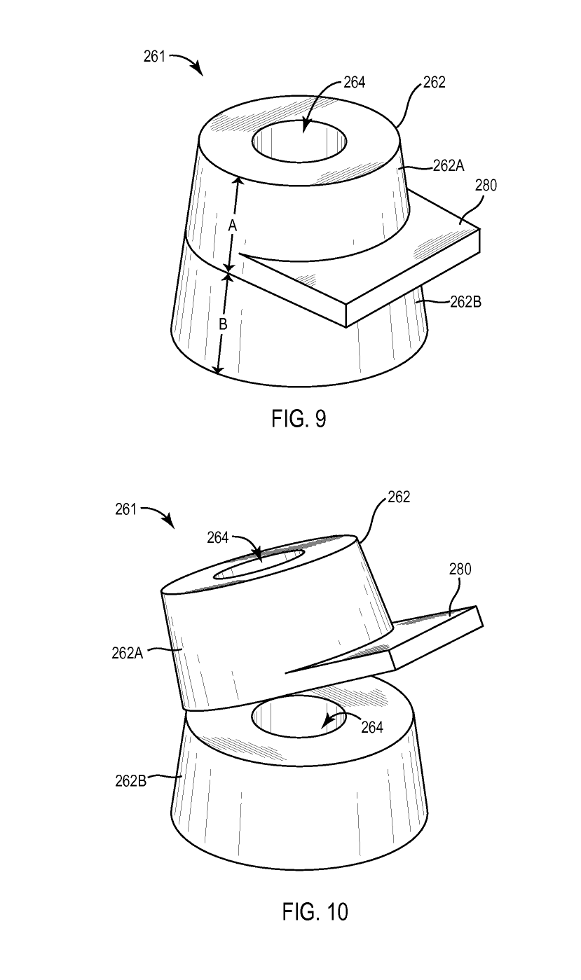

Referring now to FIGS. 9 and 10, a biopsy valve 261 includes a body 262 and a planar tab 280. The biopsy valve 262 may comprise one or more materials that are thermoset or thermoplastic and/or which are elastomeric. In one embodiment, the biopsy valve 262 is made of silicone (or compositions thereof) or thermoplastic elastomer, which exhibits similar elastic properties to silicone. In one embodiment, the body 262 may have a frustoconical shape. In other embodiments, not illustrated, the body 262 may have another shape, for example, a cylindrical shape.

The body 262 is laterally divided into two portions: an upper portion 262A having a height A and a lower portion 262B having a height B. At the location of the perforation forming the lateral division, a bottom face of the upper portion 262A faces a top face of the lower portion 262B. The upper portion 262A is biased in a closed position (See FIG. 9) by a suction in the biopsy port 60 (see FIG. 1). In some embodiments, the height A and the height B are equal. In other embodiments, the height A is greater than the height B. In additional embodiments, the height A is less than the height B. In other embodiments, the perforation that forms the lateral division may be provided at a predetermined location such that a ratio of the height A to the height B is within a predetermined range. The body 262 further includes a through hole 264 extending along a central, longitudinal axis of the body 262.

The upper portion 262A and the lower portion 262B are hingedly connected, with the upper portion 262A being able to rotate about the hinge and move relative to the lower portion 262B, which is generally stationary. In some embodiments, the biopsy valve 261 includes a tab 280 configured to assist the endoscopist in moving the upper portion 262A from the closed position (see FIG. 9) to an open position (see FIG. 10). When the upper portion 262A is in the open position, the endoscopist can release suction in the biopsy port 60 through a side-vent without removing the entire biopsy valve 261 (see FIG. 6) or opening the lid of the biopsy valve 261 (see FIG. 5).

In order to allow the upper portion 262A to reversibly move between the closed position and the open position, the upper portion 262A and the lower portion 262B are hingedly connected. In one example, the upper portion 262A and the lower portion 262B are hingedly connected by a living hinge provided on a portion of an outer circumference of the upper portion 262A and the lower portion 262B.

In some embodiments, the biopsy valve 261 may include a bi-stable hinge having a thin hinge web having two, spaced apart pivot axes or hinges, one hinge connecting the upper portion 262A to the hinge web and the other hinge connecting the lower portion 262B to the hinge web. The hinge web may be a living hinge. The bi-stable hinge is biased closed and only a small amount of force is needed to shift from a closed position to an open position.

The tab 280 is provided at a position approximately 180 degrees from the hinge, with respect to an outer circumference of the body 262. The tab 280 protrudes through the body 262 such that a first portion of the tab 280 is located at an exterior of the body 262 and a second portion of the tab 280 is located at an interior of the body 262. The tab 280 is configured such that pushing up on the first portion of the tab 280 raises the upper portion 262A to an open position. Because the upper portion 262A is normally biased in the closed position, once the upward force exerted on the first portion of the tab 280 is removed (i.e., once the endoscopist is no longer pushing up on the first portion of the tab 280), the upper portion 262A returns to the closed position. The upper portion 262A can be reversibly moved from the closed position to the open position, for example, by the endoscopist's thumb such that the endoscopist can use the same hand (i.e., one hand) to operate the endoscope 100 and move the biopsy valve 261 from the closed position to the open position to release suction through the side-vent. This cannot be done for a conventional biopsy valve 61 for which the suction release techniques (illustrated in FIGS. 5 and 6) require two hands to perform.

In one embodiment, the second portion of the tab 280 is only provided in a region extending from the outer circumference of the upper portion 262A or the lower portion 262B to the through hole 264 extending along the central, longitudinal axis of the body 262. In the case where the second portion of the tab 280 is only provided in this region, a slight gap may be formed between the upper portion 262A and the lower portion 262B. In some embodiments, this gap may be eliminated by disposing the second portion of the tab 280 in a recess provided in the bottom face of the upper portion 262A (or, alternatively, in the top face of the bottom portion 262B) such that a bottom face of the second portion of the tab 280 is planar with the bottom face of the upper portion 262A (or, alternatively, such that a top face of the second portion of the tab 280 is planar with the upper face of the lower portion 262B). In other embodiments, the gap may be sealed, by a flange protruding from the upper portion 262A or the lower portion 262B.

In another embodiment, the second portion of the tab 280 may have a same surface area as a surface area of the bottom face of the upper portion 262A. In this embodiment, a center of the second portion of the tab 280 includes an aperture having the same dimensions as the through hole 264, such that biopsy forceps or other tools may still be passed through the biopsy port 60.

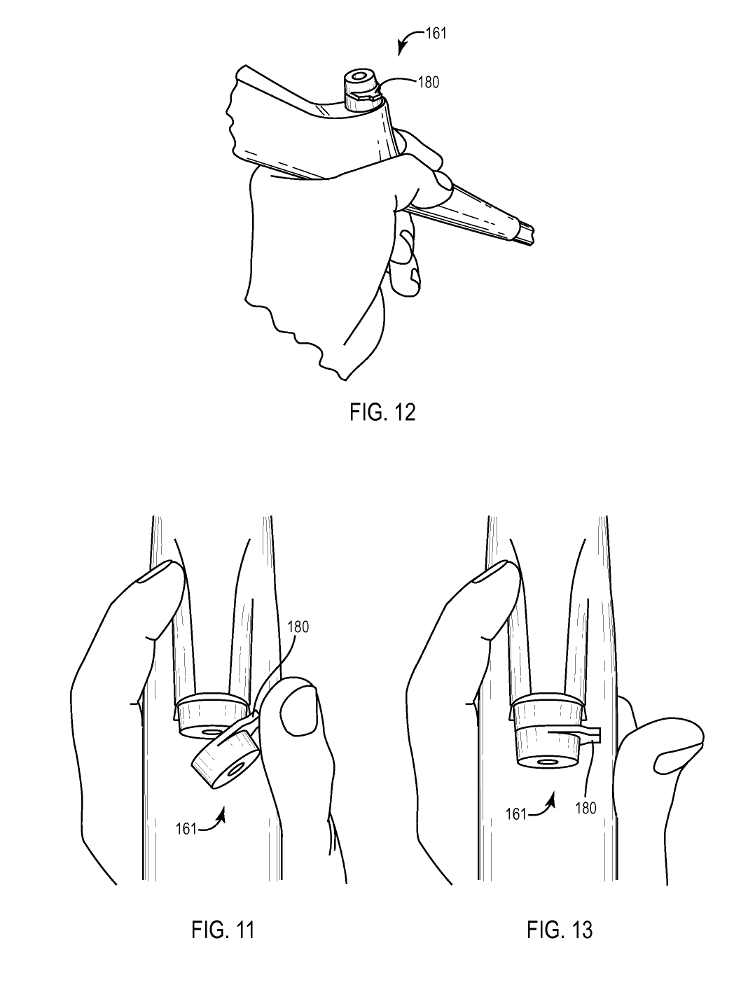

In some embodiments, the lid of the biopsy valve may be eliminated altogether, while in other embodiments the lid of the biopsy valve may be retained. Although FIGS. 7-10 do not illustrate a lid on the biopsy valve, one of ordinary skill in the art would appreciate that it is possible to place a tethered lid on the biopsy valves illustrated in FIGS. 7-10. Similarly, while the biopsy valves illustrated in FIGS. 11-13 include a lid, one of ordinary skill in the art would appreciate that it is possible to eliminate the lid from the biopsy valve. When the biopsy valve does not include a lid, one of ordinary skill in the art would appreciate that to maintain suction, an entrance to the through hole must be sealed. However, the seal must be capable of being penetrated by biopsy forceps or other tools, while maintaining suction in the biopsy port.

In the embodiments described above, the biopsy valve is biased in a closed position. However, in other applications, it may be beneficial for the biopsy valve to be biased in an open position. One of ordinary skill in the art would appreciate that the biopsy valve described in the embodiments above can be configured to be biased in either a closed position or an open position or to be biased to a closed position and an open position via a bi-stable design.

By utilizing a biopsy valve described in the embodiments above, an endoscopist's efficiency is increased and an endoscopic procedure is made easier by decreasing the amount of time the endoscopist spends removing and replacing the lid of the biopsy valve. A likelihood of fecal material or other bodily material being ejected from the uncovered biopsy port onto the patient or any individuals in the area in which the endoscopic procedure is being performed is also decreased. This reduces the risk of infection and/or contamination. The improvement in efficiency and the decreased risk of fecal material or other bodily material ejection is cumulative for each time the lid of the biopsy valve or the entire biopsy valve would have had to been removed, which may occur between 5-10 times per procedure. In some embodiments, the lid of the biopsy valve may be eliminated altogether, while in other embodiments the lid of the biopsy valve may be retained. Although FIGS. 7-10 do not illustrate a lid on the biopsy valve, one of ordinary skill in the art would appreciate that it is possible to place a tethered lid on the biopsy valves illustrated in FIGS. 7-10. Similarly, while the biopsy valves illustrated in FIGS. 11-13 include a lid, one of ordinary skill in the art would appreciate that it is possible to eliminate the lid from the biopsy valve. When the biopsy valve does not include a lid, one of ordinary skill in the art would appreciate that to maintain suction, an entrance to the through hole must be sealed. However, the seal must be capable of being penetrated by biopsy forceps or other tools, while maintaining suction in the biopsy port.

The construction and arrangements of the endoscopic side release biopsy valve, as shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, image processing and segmentation algorithms, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present invention.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

What is claimed is:

1. A biopsy valve for an endoscope comprising:
a valve body that is laterally divided into an upper portion having a first height and a lower portion having a second height, the upper portion and the lower portion being hingedly connected such that the upper portion is configured to rotate about a hinge and move relative to the lower portion, which is generally stationary;
a through hole provided along a central, longitudinal axis of the valve body; and
a tab disposed between the upper portion of the valve body and the lower portion of the valve body,
wherein the upper portion is biased in a closed position in which suction is maintained in the endoscope, and
wherein exerting an upward or a downward force on the tab rotates the upper portion about the hinge into an open position in which suction is released in the endoscope.

2. The biopsy valve of claim 1, wherein the tab has a first portion exterior to the valve body and a second portion interior to the valve body.

3. The biopsy valve of claim 2, wherein the second portion of the tab is only provided in a region extending from the hinge to the through hole.

4. The biopsy valve of claim 2, wherein the second portion of the tab is provided in a recess disposed in one of a bottom face of the upper portion or a top face of the lower portion such that an exposed surface of the tab is planar with a surface of the upper portion or a surface of the lower portion closest to a lateral division of the valve body.

5. The biopsy valve of claim 2, wherein the second portion of the tab has a same surface area as a surface area of a face of the upper portion or the lower portion to which the tab is connected, and wherein the second portion of the tab includes a central aperture having a same diameter as a diameter of the through hole.

6. The biopsy valve of claim 1, wherein the tab is provided at a position located approximately 180 degrees from the hinge, with respect to an outer circumference of the valve body.

7. The biopsy valve of claim 1, wherein a gap formed between the upper portion and the lower portion is sealed by a flange protruding from one of the upper portion and the lower portion.

8. The biopsy valve of claim 1, wherein the hinge is a living hinge.

9. The biopsy valve of claim 1, further comprising a lid tethered to the upper portion, the lid configured to seal the through hole.

10. An endoscope configured for use in an endoscopic procedure, the endoscope comprising:
a biopsy port;
a suction port configured to converge with the biopsy port and maintain suction in the biopsy port; and
a biopsy valve configured to close off a proximal opening of the biopsy port and prevent room air from being drawn into the suction port, the biopsy valve including
a valve body that is laterally divided into an upper portion having a first height and a lower portion having a second height, the upper portion and the lower portion being hingedly connected such that the upper portion is configured to rotate about a hinge and move relative to the lower portion, which is generally stationary;
a through hole provided along a central, longitudinal axis of the valve body; and
a tab disposed between the upper portion of the valve body and the lower portion of the valve body,
wherein the upper portion is biased in a closed position in which suction is maintained in the biopsy port, and
wherein exerting an upward or a downward force on the tab rotates the upper portion about the hinge into an open position in which suction is released in the biopsy port.

11. The endoscope of claim 10, wherein the tab has a first portion exterior to the valve body and a second portion interior to the valve body.

12. The endoscope of claim 11, wherein the second portion of the tab is only provided in a region extending from the hinge to the through hole.

13. The endoscope of claim 11, wherein the second portion of the tab is provided in a recess disposed in one of a bottom face of the upper portion or a top face of the lower portion such that an exposed surface of the tab is planar with a surface of the upper portion or a surface of the lower portion closest to a lateral division of the valve body.

14. The endoscope of claim 11, wherein the second portion of the tab has a same surface area as a surface area of a face of the upper portion or the lower portion to which the tab is connected, and wherein the second portion of the tab includes a central aperture having a same diameter as a diameter of the through hole.

15. The endoscope of claim 10, wherein the tab is provided at a position located approximately 180 degrees from the hinge, with respect to an outer circumference of the valve body.

16. The endoscope of claim 10, wherein a gap formed between the upper portion and the lower portion is sealed by a flange protruding from one of the upper portion and the lower portion.

17. The endoscope of claim 10, wherein the hinge is a living hinge.

18. The endoscope of claim 10, further comprising a lid tethered to the upper portion, the lid configured to seal the through hole.

19. A method of operating a biopsy valve of an endoscope, the biopsy valve including a valve body that is laterally divided into an upper portion having a first height and a lower portion having a second height, the upper portion and the lower portion being hingedly connected such that the upper portion is configured to rotate about a hinge and move relative to the lower portion, which is generally stationary, a through hole provided along a central, longitudinal axis of the valve body, and a tab disposed between the upper portion of the valve body and the lower portion of the valve body, the method comprising:
exerting an upward or a downward force on the tab to rotate the upper portion about the hinge from a closed position in which suction is maintained in the endoscope to an open position in which suction is released in the endoscope; and
removing the upward or the downward force on the tab to reversibly rotate the upper portion about the hinge to the closed position.

20. The method of claim 19, wherein the upward or the downward force is exerted on the tab by a same hand used to operate the endoscope.
